(19) United States
(12) Patent Application Publication
(10) Pub. No.: US 2025/0319250 A1
(43) Pub. Date: Oct. 16, 2025

(54) ARTIFICIAL INTELLIGENCE DRIVEN AUTOMATED INFUSION PUMP FOR ADMINISTERING INTRAVENOUS MEDICATION INFUSIONS

(71) Applicant: DyQure LLC, Sauk Rapids, MN (US)

(72) Inventors: Adnan I. Qureshi, Columbia, OH (US); Muhammad Fareed Khan Suri, Sauk Rapids, MN (US)

(21) Appl. No.: 18/631,196

(22) Filed: Apr. 10, 2024

(51) Int. Cl. A61M 5/172; A61M 5/142

(52) U.S. Cl. CPC A61M 5/1723; A61M 5/14212; A61M 2205/3331; A61M 2205/505; A61M 2205/52; A61M 2230/04; A61M 2230/30

(57) ABSTRACT

Apparatuses, systems, and methods employing a machine learning algorithm to manage patient blood pressure. The algorithm uses recorded blood pressure from real time monitoring of a patient with a particular disease and target blood pressure and calculates the magnitude of blood pressure reduction over time. An appropriate dose of an intravenous antihypertensive medication infusion is calculated initially from an algorithm that uses data from numerous previous patients to estimate the required dose adjustment most likely to achieve the desired magnitude of blood pressure reduction. The system can create a new algorithm based on blood pressure response to particular dose adjustments in the patient being treated and, after internal validation, switches to the new algorithm to estimate the required dose adjustment most likely to achieve the desired magnitude of blood pressure reduction.

### TECHNICAL FIELD

The invention relates to devices and methods for prevention and treatment of stroke, and more specifically, apparatuses and methods for treating intracranial arterial diseases that cause of exacerbate ischemic stroke.

### BACKGROUND

Infusions of medications to treat blood pressure in intensive care units are often suboptimal. Excessive delay in reaching the treatment goal and fluctuations in blood pressure are common, and lead to suboptimal patient outcomes. See Toyoda K, Koga M; as the SAMURAI Investigators. Controlling blood pressure soon after intracerebral hemorrhage: The SAMURAI-ICH Study and its successors. Hypertens Res. 2022; 45(4):583-590. doi: 10.1038/s41440-022-00866-8; Rabinstein A A. Optimal Blood Pressure After Intracerebral Hemorrhage: Still a Moving Target. Stroke. 2018; 49(2):275-276. doi: 10.1161/STROKEAHA.117.020058; Koga M, Arihiro S, Hasegawa Y, et al. Intravenous nicardipine dosing for blood pressure lowering in acute intracerebral hemorrhage: the Stroke Acute Management with Urgent Risk-factor Assessment and Improvement-Intracerebral Hemorrhage study. J Stroke Cerebrovase Dis. 2014; 23(10):2780-2787. doi: 10.1016/j.jstrokecerebrovasdis.2014.06.029; de Havenon A, Majersik J J, Stoddard G, et al. Increased Blood Pressure Variability Contributes to Worse Outcome After Intracerebral Hemorrhage. Stroke. 2018; 49(8):1981-1984. doi:10.1161/STROKEAHA.118.022133; Qureshi A L, Palesch Y Y, Foster L D, et al. Blood Pressure-Attained Analysis of ATACH 2 Trial, Stroke. 2018; 49(6):1412-1418. doi:10.1161/STROKEAHA.117.019845.

One problem in prior practice is that the response to blood pressure is not automated and requires manual interpretation of data. A nurse may have to look at the data and decide on the appropriate change in medication. The minimum time interval for patient evaluation in the intensive care unit is once every 30 minutes, which can lead to delays in responding to particular abnormal values.

Another problem is that the response to an abnormal value is typically not specific to a disease process, and is based on pre-existing algorithms derived from other disease processes. There is no learning from previous responses in a particular patient that is incorporated into the next infusion medication change. Currently, there is judgment applied by the treating nurse, but nurses change every 8-12 hours, and the incoming person is often unaware of the dose-response relationship observed in the previous shift.

Therefore, what is needed are devices, systems, and methods that reduce the need for manual tracking and interpretation of data, that can provide guidance as to appropriate dosage rates for infusion-delivered medication, and that can use patient-specific data learned during the course of treatment as well as the large volumes of data gained during the course of treatment of other patients with similar conditions to accomplish this.

### SUMMARY

According to embodiments of the invention, a system uses recorded blood pressure from a real time monitoring of a patient with a particular disease and target blood pressure and calculates the magnitude of blood pressure reduction over time. An appropriate dose of an intravenous antihypertensive medication infusion is calculated initially from an algorithm that uses data from numerous previous patients to estimate the required dose adjustment most likely to achieve the desired magnitude of blood pressure reduction. The system displays the recommended dose adjustment on the screen for operator to accept. Once the operator accepts, the system dispenses the dose via an automated infusion system. The system creates a new algorithm based on blood pressure response to particular dose adjustments in the patient being treated and, after internal validation, switches to the new algorithm to estimate the required dose adjustment most likely to achieve the desired magnitude of blood pressure reduction.

In an embodiment, an automated system for managing blood pressure of a patient includes a blood pressure measuring device and a controller communicatively coupled with the blood pressure measuring device. The controller includes a processor programmed with a machine learning algorithm, the algorithm adapted to receive a systolic blood pressure measurement from the blood pressure measuring device, perform a probabilistic determination to predict a blood pressure response to a particular dose of an intravenous medication and develop a dosage recommendation to achieve a predetermined target systolic blood pressure goal; a memory adapted to store a plurality of data elements including data relating to the intravenous medication, data relating to the patient, data relating to a plurality of hemodynamic parameters, and data relating to other patients with a same or a similar condition; and a user interface including a display. The system further includes an infusion pump apparatus communicatively coupled with the controller. The infusion pump apparatus includes an infusion pump, a medication bag fluidly coupled with the infusion pump, and an intravenous port fluidly coupled with the infusion pump. The machine learning algorithm uses at least one of the plurality of data elements to develop the dosage recommendation, the controller presents the dosage recommendation to an operator using the user interface, and the controller instructs the infusion pump to implement the dosage recommendation.

In embodiments, the data relating to the medication includes at least one of known pharmacology of the medication, historic dose response data for the current patient, and last dose change duration for the current patient. The data relating to a plurality of hemodynamic parameters can include at least one of patient heart rate, patient stroke volume, patient cardiac output, and patient total peripheral resistance.

In embodiments, the machine learning algorithm of the system automatically alters itself based on patient blood pressure response to the medication after a predetermined number of sequential dose adjustments, and develops the dosage recommendation according to the altered algorithm.

In embodiments, the system can further include a second infusion pump apparatus communicatively coupled with the controller, the second infusion pump apparatus comprising a second infusion pump and a second medication bag fluidly coupled with the second infusion pump, the first infusion pump apparatus dispensing a first medication and the second infusion pump apparatus dispensing a second medication different from the first medication, the algorithm being further adapted to perform a probabilistic determination to predict a blood pressure response to a particular dose of each of the first and second medications and develop a dosage recommendation for each of the first and second medications to achieve the predetermined target systolic blood pressure goal. A heart rate sensor or an intracranial pressure sensor can be communicatively coupled to the controller.

In embodiments, the display of the user interface of the system is touch sensitive and presents icons enabling the operator to accept, reject, or adjust the dosage recommendation.

In further embodiments, an apparatus for determining an optimal intravenous medication dosage to manage blood pressure in a patient includes a blood pressure measuring device and a controller communicatively coupled with the blood pressure measuring device. The controller includes a processor programmed with a machine learning algorithm, the algorithm adapted to receive a systolic blood pressure measurement from the blood pressure measuring device, perform a probabilistic determination to predict a blood pressure response to a particular dose of an intravenous medication and develop a dosage recommendation to achieve a predetermined target systolic blood pressure goal; a memory adapted to store a plurality of data elements including data relating to the intravenous medication, data relating to the patient, data relating to a plurality of hemodynamic parameters, and data relating to other patients with a same or a similar condition; and a user interface including a display. The machine learning algorithm uses at least one of the plurality of data elements to develop the dosage recommendation, and the controller presents the dosage recommendation to an operator using the user interface.

In embodiments, the apparatus further includes an infusion pump apparatus communicatively coupled with the controller and comprising an infusion pump, a medication bag fluidly coupled with the infusion pump, and an intravenous port fluidly coupled with the infusion pump, wherein the controller instructs the infusion pump to implement the dosage recommendation.

In embodiments, the data relating to the medication includes at least one of known pharmacology of the medication, historic dose response data for the current patient, and last dose change duration for the current patient. The data relating to a plurality of hemodynamic parameters can include at least one of patient heart rate, patient stroke volume, patient cardiac output, and patient total peripheral resistance.

In embodiments, the machine learning algorithm of the apparatus automatically alters itself based on patient blood pressure response to the medication after a predetermined number of sequential dose adjustments, and develops the dosage recommendation according to the altered algorithm.

In other embodiments, the apparatus further includes a second infusion pump apparatus communicatively coupled with the controller, the second infusion pump apparatus including a second infusion pump and a second medication bag fluidly coupled with the second infusion pump, the first infusion pump apparatus dispensing a first medication and the second infusion pump apparatus dispensing a second medication different from the first medication, the algorithm being further adapted to perform a probabilistic determination to predict a blood pressure response to a particular dose of each of the first and second medications and develop a dosage recommendation for each of the first and second medications to achieve the predetermined target systolic blood pressure goal.

In embodiments, the apparatus further includes a heart rate sensor or an intracranial pressure sensor communicatively coupled to the controller. The display of the user interface may be touch sensitive and present icons enabling the operator to accept, reject, or adjust the dosage recommendation.

In further embodiments, a method of dispensing intravenous medication includes measuring a patient blood pressure, communicating the measured patient blood pressure to a controller having a processor programmed with a machine learning algorithm, the algorithm adapted to receive the measured patient blood pressure, perform a probabilistic determination to predict a blood pressure response to a particular dose of an intravenous medication and develop a dosage recommendation to achieve a predetermined target systolic blood pressure goal, the controller having a memory adapted to store a plurality of data elements including data relating to the intravenous medication, data relating to the patient, data relating to a plurality of hemodynamic parameters, and data relating to other patients with a same or a similar condition, wherein the machine learning algorithm uses at least one of the plurality of data elements to develop the dosage recommendation, presenting the dosage recommendation to an operator through a user interface including a display, and implementing the dosage recommendation with a first infusion pump apparatus communicatively coupled with the controller.

In embodiments, the method can further include presenting the operator with controls enabling the operator to accept, reject, or alter the dosage recommendation. The method can further include providing a second infusion pump apparatus communicatively coupled with the controller, the first infusion pump apparatus dispensing a first medication and the second infusion pump apparatus dispensing a second medication different from the first medication, the algorithm being further adapted to perform a probabilistic determination to predict a blood pressure response to a particular dose of each of the first and second medications and develop a dosage recommendation for each of the first and second medications to achieve the predetermined target systolic blood pressure goal, and implementing each of the dosage recommendations for the first and second medications.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1 is a generalized schematic depiction of an artificial intelligence driven automated infusion pump system according to an embodiment of the invention;

FIG. 2 is a schematic depiction of a machine learning process for an artificial intelligence driven automated infusion pump system according to an embodiment of the invention;

FIG. 3 is a schematic depiction of a machine learning process for an artificial intelligence driven automated infusion pump system according to another embodiment of the invention;

FIG. 4 is a schematic depiction of a machine learning process for an artificial intelligence driven automated infusion pump system according to another embodiment of the invention;

FIG. 5 is a schematic depiction of a menu-driven display for an artificial intelligence driven automated infusion pump system according to an embodiment of the invention;

FIG. 6 is a schematic depiction an artificial intelligence driven automated infusion pump apparatus according to an embodiment of the invention;

FIG. 7 is a schematic depiction of an artificial intelligence driven automated infusion pump apparatus according to another embodiment of the invention; and

FIG. 8 is a schematic depiction of an artificial intelligence driven automated infusion pump apparatus according to another embodiment of the invention.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE DRAWINGS

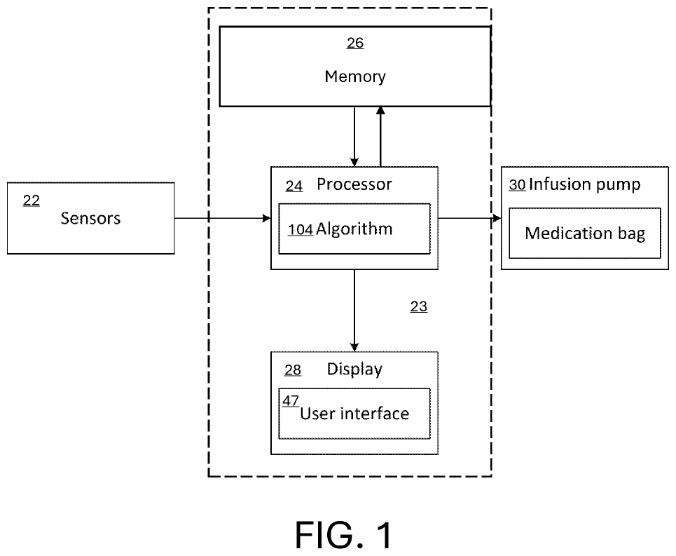

In FIGS. 1 and 6-8 there are depicted schematics of an artificial intelligence driven automated infusion pump apparatus 20 according to embodiments of the invention. Apparatus 20 generally includes sensors 22, controller 23 which includes processor 24, memory 26, and display 28, and infusion pump assembly 30. Sensors 22 generally include blood pressure measuring device 32, and can also include various other sensors, such as intracranial pressure sensor 34, heart rate monitoring device 36, or sensors for any other measurable attribute that may be necessary or useful for determining dosing of infused medication or treatment of a patient condition. Blood pressure measuring device 32 can be any device for automated measurement of patient blood pressure as is known in the industry, and may have circuitry enabling variably timed measurements at intervals selected manually by a user, or selected via data link from an external source.

Processor 24 can be any general or special purpose electronic processor programmed to run algorithm(s) 104, the operation of which will be further described hereinbelow. Memory 26 can generally include any type of electronic memory compatible with processor 24, such as for example, random access memory modules, read-only memory modules, and mechanical or solid-state disk storage.

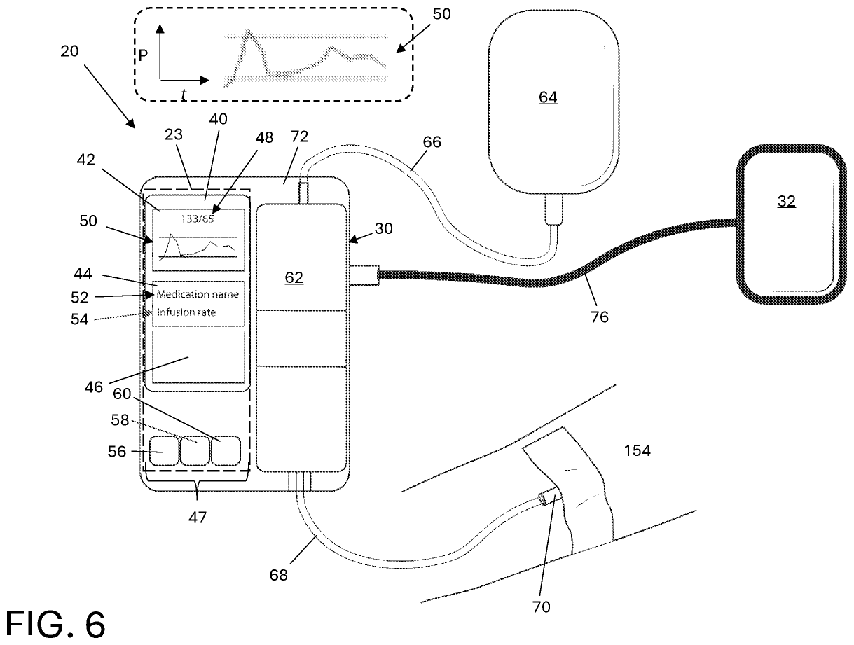

As depicted in FIGS. 6-8, display 28 generally includes display screen 40, which can include blood pressure information display 42, medication information display 44, dosage information display 46, and user interface 47. Blood pressure information display 42 may include the last measured systolic/diastolic pressure reading 48, typically a systolic/diastolic value in mm Hg (e.g., 133/65 mm Hg), and may also include a graphical display 50. As depicted in the inset of FIG. 6, graphical display 50 can include a graph of historic measured blood pressure (P) over time (t) and with lower and upper lines denoting a targeted systolic blood pressure range (e.g., 110-140 mm Hg). Medication information display 44 can include medication name 52, current infusion rate 54, and any other medication related information that may be useful to an operator. Dosage information display 46 can display information such as for example a suggested new dose calculated by algorithm 104 and the time elapsed since the last dosage change. User interface 47 can generally include accept dose button 56, reject dose button 58, and change dose button 60 for accepting operator input. It will be appreciated that accept dose button 56, reject dose button 58, and change dose button 60, may be separate push buttons as depicted or may be integrated as touch-sensitive screen icons on display screen 40 as will be described further hereinbelow.

Infusion pump assembly 30 generally includes infusion pump 62, medication bag 64, inlet tube 66, outlet tube 68, and intravenous port 70. Infusion pump 62 can be any known electronically controllable infusion pump as is commonly known in the art. Such pumps typically operate by constricting or relaxing outlet tube 68, depending on the desired dosage rate.

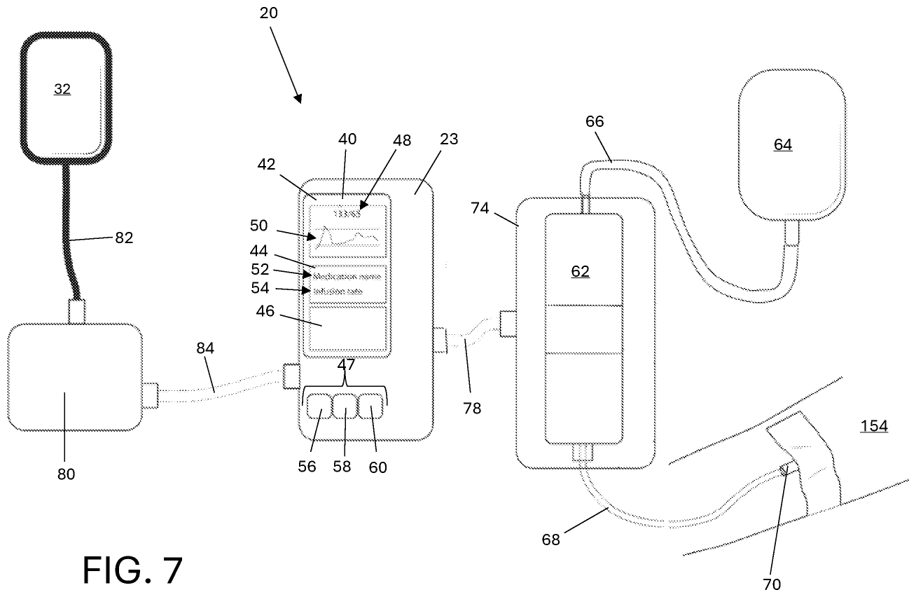

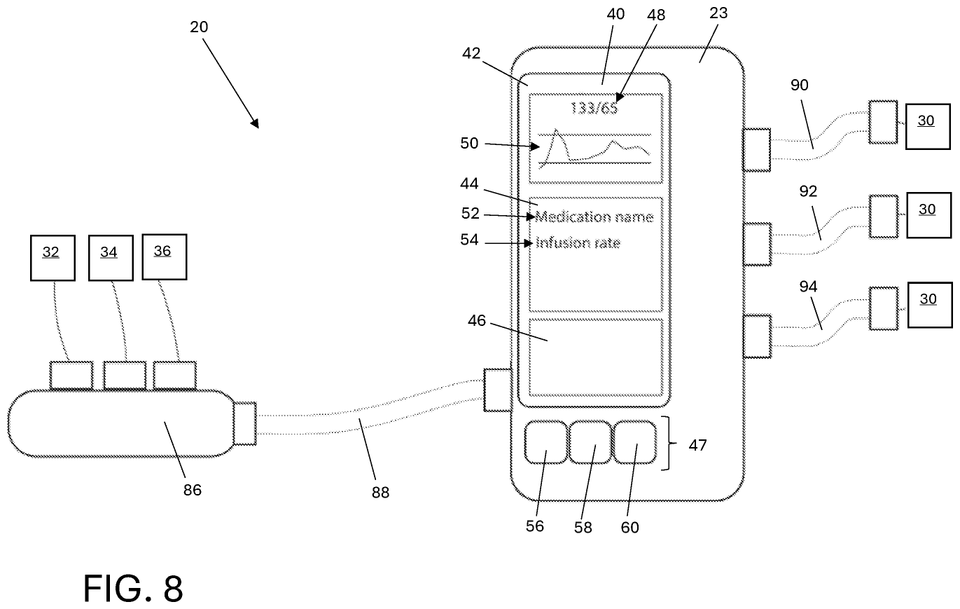

As depicted in FIG. 6, infusion pump 62 and controller 23 may be contained in a common housing 72. Alternatively, infusion pump 62 may be contained in its own separate housing 74 as depicted in FIGS. 7 and 8. In the embodiment of FIG. 6, controller 23 and infusion pump 62 are communicatively coupled inside housing 72, and blood pressure measuring device 32 is communicatively coupled with controller 23 through data cable 76. In the embodiment of FIG. 7, controller 23 and infusion pump 62 are communicatively coupled though data cable 78. Blood pressure recording device 80 receives blood pressure measurements through data cable 82, and communicates blood pressure data to controller 23 through data cable 84. In the embodiment of FIG. 8, interface 86 receives inputs from blood pressure measuring device 32, intracranial pressure sensor 34, and heart rate monitoring device 36, and aggregates and communicates the measurements to controller 23 through data cable 88. It will be appreciated that other parameters could also be sensed and communicated through interface 86 if desired or needed. Multiple infusion pumps 30, each dispensing a separate medication, are communicatively coupled to controller 23 through data cables 90, 92, 94. It will be appreciated that any or all of data cables 76, 78, 82, 84, 90, 92, and 94 could be replaced with wireless communications such as for example WiFi connections as are known in the art. As depicted, any of the embodiments depicted in FIGS. 6-8 are compact and can be arranged at the bedside of a patient using a suitable stand or rack.

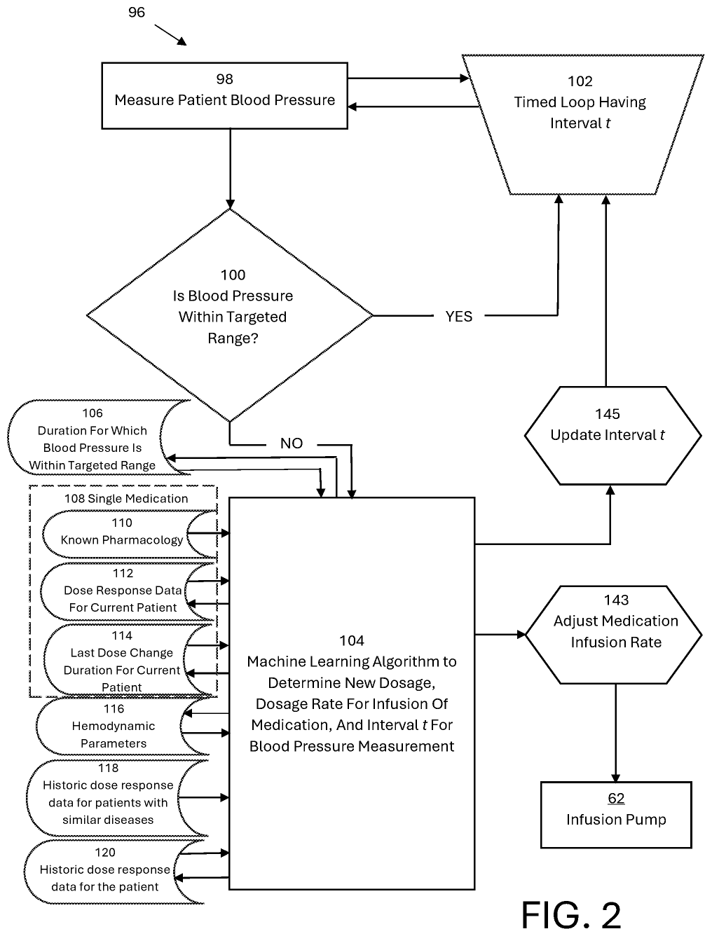

In FIG. 2, an embodiment of a process and algorithm for an artificial intelligence driven infusion pump system 96 is schematically depicted. The first step is measurement of patient blood pressure 98, performed using blood pressure measuring device 32. The digitalized output of blood pressure measuring device 32 communicated to controller 23 may be internally validated to detect possible erroneous readings. Any suspected erroneous readings cause controller 23 to request a repeat measurement before proceeding.

Once an accurate blood pressure reading is achieved, controller 23 determines at step 100 whether the blood pressure reading is within a targeted range predetermined by the operator. For example, the operator could specify that systolic pressure must be between 100 and 140 mm/Hg. If yes, then blood pressure measuring device 32, which may operate on a timed loop 102 having variable time interval t, may be permitted to proceed with the next timed measurement of blood pressure at the set time interval t. Variable time interval t can be any suitable time period, but typically ranges from about 5 to 10 minutes.

If no at step 100, then the blood pressure reading from blood pressure measuring device 32 is communicated to machine learning algorithm. 104 which runs on processor 24. Machine learning algorithm 104 performs a probabilistic determination using a Bayesian, random forest, and/or decision tree methodology to predict response to a particular dose of intravenous medication. Other models such as linear parametric models or optimal discriminant analysis to determine the degree of dose can be distinguished based on distribution of response (e.g., blood pressure) and may be used to determine the optimal dose. Machine learning algorithm 104 enables controller 23 to make this probabilistic determination by considering a large amount of data together and identifying the patterns in the data (e.g., change in blood pressure expected with a particular dose and rate given the existing blood pressure). Further, machine learning algorithm 104 may be altered from its original settings based on collected data over the last few hours.

Moreover, a smart BP PID algorithm can be employed. This algorithm operates on the principles of a Proportional-Integral Derivative (PID) control system, leveraging previous blood pressure values to dynamically adjust medication dosage and maintain blood pressure within a specified range. It incorporates patient-specific sensitivity to the administered drug, dynamically adjusting the dose step size as a proportional constant within the PID framework. By monitoring blood pressure over dynamic intervals and analyzing the patient's response to medication, the algorithm autonomously corrects deviations from the target range, optimizing medication dosage in real-time. This approach offers a sophisticated yet adaptable solution for hypertensive patient management, ensuring precise and personalized treatment while minimizing the risk of adverse effects.

Different patient groups may be identified based on prognostically important characteristics such as, for example, patient age, and machine learning algorithm 104 may use group specific data to create population specific algorithms for patients within each group. For example, machine learning algorithm 104 derived from numerous patients with intracerebral hemorrhage may have identified that a dose increment of 5 mg/hr of a particular antihypertensive medication is most likely to achieve a particular target systolic blood pressure reduction. This dose recommendation for an intravenous medication to reduce blood pressure may require integration of more than one physiological measurement such as heart rate and/or intracranial pressure to avoid tachycardia or intracranial hypertension, depending on the pharmacological characteristics and adverse side effects of the medication.

As depicted in FIG. 2, pertaining to infusion of a single medication, various data stores hosted in memory 26 can provide input to machine learning algorithm 104. Data store 106 contains the durations for which patient blood pressure has historically been within the operator specified targeted range, and is updated with each blood pressure measurement iteration. Data store 108 pertains to data regarding the specific medication being dispensed, and generally includes known pharmacology 110 of the medication, historic dose response data 112 for the current patient, and last dose change duration 114 for the current patient. As depicted, dose response data 112 and last dose change duration 114 are updated with each blood pressure measurement iteration. Data store 116 contains other hemodynamic parameters in addition to the primary hemodynamic parameters of blood pressure and heart rate, such as for example, stroke volume (SV), cardiac output (CO), and total peripheral resistance (TPR). Data store 118 contains historic dose response data for the medication being dispensed for patients with similar diseases, and may include a large database compiling such data. Data store 120 contains historic dose response data for the patient being treated, and can be updated with each blood pressure measurement iteration. It will be appreciated that, although not depicted in FIG. 2, measurements of intracranial pressure and/or heart rate may be supplied to controller 23 to enable machine learning algorithm 104 to take those parameters into account when determining an appropriate dosage recommendation.

For an example in use, a patient with intracerebral hemorrhage may present with a measured systolic blood pressure of 170 mm Hg. Machine learning algorithm 104 first assesses the accuracy of the blood pressure measurement using pre-defined criteria. Based on those criteria, the measurement will be accepted as accurate, or a repeat measurement will be performed. Once an accurate systolic blood pressure measurement of 170 mm Hg is received, machine learning algorithm 104 compares that measurement with a desired systolic blood pressure which, for example, has been previously specified as <140 mm Hg by the operator. The current accurate blood pressure reading and range specified by the operator is displayed on blood pressure information display 42 as depicted in FIG. 6. Current information about the medication and dosage rate is displayed on medication information display 44. In the example, machine learning algorithm 104 will identify that a systolic blood pressure reduction of 30 mm Hg is the goal. Machine learning algorithm 104 then uses the information in data stores 106, 108, 116, 118 and 120 to determine the most appropriate dose and rate of intravenous medication to achieve the target blood pressure goal by using machine learning models as described above.

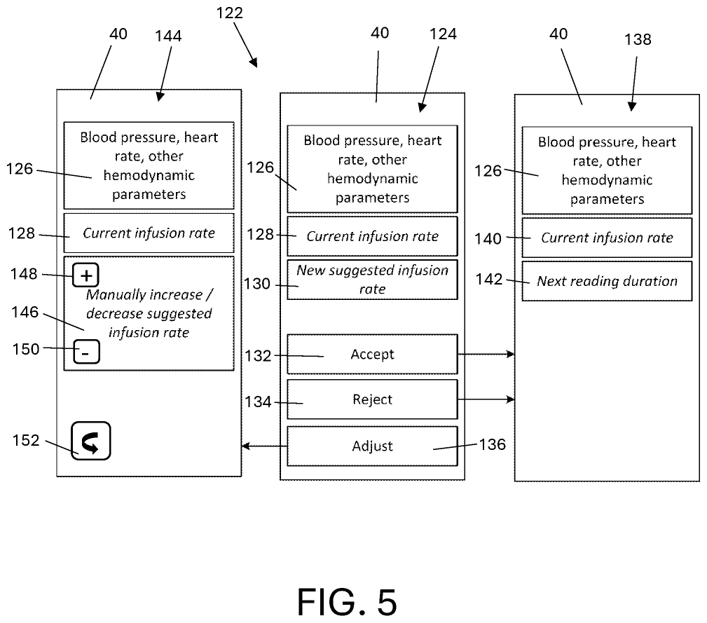

FIG. 5 depicts an embodiment of a user interface 122 implemented on display screen 40. User interface 122 can be used with any of the embodiments of FIGS. 6-8. Display screen 40 can be implemented using known capacitive touch-screen technology, or any other similar technology. Main menu 124, which is the initial default menu displayed, generally includes hemodynamic information block 126, where information such as the last accurate systolic/diastolic blood pressure reading, heart rate, and other hemodynamic parameters such as SV, CO, and TPR can be displayed. In addition, a graphical representation of blood pressure over time with selected limits such as graphical display 50 depicted in FIGS. 6-8 can be displayed in hemodynamic information block 126. Medication information block 128 displays the current infusion rate for the medication, along with the medication name and any other such medication related information. Information block 130 displays a new suggested infusion rate as suggested by machine learning algorithm 104. Touch sensitive icons or buttons 132, 134, 136, enable an operator to interact with the apparatus. If the operator selects “ACCEPT” by pressing icon or button 132, meaning the new suggested infusion rate is to be implemented, menu 138 is displayed, and it includes hemodynamic information block 126 as before, while information block 140 displays the new infusion rate for the medication, along with the medication name and any other such medication related information. Duration information block 142 displays the duration until the next scheduled blood pressure measurement, at which time the process and algorithm will be performed again. Referring again to FIG. 2, the medication infusion rate is adjusted at step 143 with an appropriate instruction to infusion pump 62. The interval/for the next blood pressure measurement can also be adjusted at step 145 and communicated to blood pressure measuring device 32 if requested by machine learning algorithm 104 or set manually.

If “REJECT” is selected by pressing icon or button 134, menu 138 is also displayed, except that the infusion rate is not changed, the current infusion rate is displayed in block 140, and the system will await the next scheduled blood pressure measurement to prompt again for any suggested change. If “ADJUST” is selected by pressing icon or button 136, adjustment menu 144 is displayed on display screen 40. Hemodynamic information block 126 and Medication information block 128 are displayed as before, along with adjustment selection block 146. The previously presented suggested new dosage rate is displayed along with “+” and “−” buttons or icons 148, 150, respectively. Pressing the “+” icon or button 148 will upwardly increment the suggested dosage by a preselected amount (e.g., +0.5 mg/hr), while pressing the “.” button or icon 150 will decrement the suggested dosage by a preselected amount (e.g. −0.5 mg/hr). Buttons or icons 148, 150, can be repeatedly pressed until the desired dosage is reached. When the desired dosage is reached, the operator can press the return button or icon 152 to return to main menu 124, where the manually created dosage value will be displayed in information block 130. The operator can then proceed to accept, reject, or adjust this new value by pressing buttons or icons 132, 134, 136, respectively.

Although not depicted, main menu 124, or menus 138, 144, can also present options for selected a new reading duration wherein a new blood pressure measurement will be taken, and the process will be reinitiated. Moreover, machine learning algorithm 104 can also suggest a new reading duration (interval (of timed loop 102) which can be accepted or rejected by the operator. This suggested interval t can be similarly overridden by the operator as with the dosage increment. It will be appreciated that controller 23 can present an audible alert when the operator needs to respond to a request for acceptance, rejection, or adjustment of dosage change.

Continuing with the previous example, the recommended medication dose increment arrived at by machine learning algorithm 104 (for example 5 mg/hr) to achieve the targeted blood pressure reduction will be displayed in dosage information display 46 or information block 140. The operator can then “ACCEPT,” “REJECT,” or “ADJUST” as described above. If the operator selects “ACCEPT,” the dose recommendation will be implemented by infusion pump 62, from medication contained in medication bag 64, through inlet tube 66 and outlet tube 68, to intravenous port 70 for infusion into the bloodstream of patent 154. If the operator selects “REJECT,” the existing dosage will be continued until the next scheduled blood pressure reading 98 is taken according to interval t of timed loop 102. If the operator selects “ADJUST,” manual adjustments can be performed as described above. Alternatively, controller 23 can be set to simply implement the suggested dosage change automatically without requiring input from the operator.

A systolic blood pressure measurement 98 will be retaken according to interval t of timed loop 102 (e.g., t=5 or 10 minutes), and the process of FIG. 2 will repeat. Machine learning algorithm 104 can use 3-5 of such cycles to calculate the systolic blood pressure reduction expected with 1 mg./hr (or another dose increment) for the patients being treated. And after 5-10 dose adjustments, the machine learning algorithm 104 can self-alter to be based on the patient's own dose response data. Then, for example, once an accurate measurement of systolic blood pressure of 150 mm Hg is observed, machine learning algorithm 104 can compare with a desired systolic blood pressure (which for example may be set at <140 mm Hg according to the operator). Machine learning algorithm 104 then identifies that a systolic blood pressure reduction of 10 mm Hg is now the goal. The self-altered machine learning algorithm 104 derived from the patient's own data identifies that a dose increment of 2 mg/hr of the antihypertensive medication is most likely to achieve to this target systolic blood pressure reduction. The new dose increment of 2 mg/hr will be displayed in information block 130, and the operator options to “ACCEPT,” “REJECT” using buttons or icons 132, 134, 136, will be available.

In a more advanced embodiment, once an accurate systolic blood pressure measurement, 170 mm Hg for example, is received, machine learning algorithm 104 can compare it with a desired systolic blood pressure at a particular time (for example at 3 hours after symptom onset). This desired systolic blood pressure may be derived by machine learning algorithm 104 based on an ideal systolic blood pressure for a patient with intracerebral hemorrhage at 3 hours after symptom onset as shown by prior data. For example, machine learning algorithm 104 may determine that the ideal systolic blood pressure for a patient with intracerebral hemorrhage at 3 hours after symptom onset is <150 mm Hg. Therefore, machine learning algorithm 104 identifies that a systolic blood pressure reduction of 20 mm Hg is the goal. Then machine learning algorithm 104, using data from previously treated patients, the patient's own data, or a combination of both, may identify that a dose increment of 3 mg/hr of a particular antihypertensive medication is most likely to achieve the target systolic blood pressure reduction within a predetermined time. The dose increment of 3 mg/hr will be displayed on main menu 124 in information block 130 on display screen 40. The operator can “ACCEPT,” “REJECT,” or “ADJUST” as before. It will be appreciated that any number of such temporal target thresholds can be used within the system to conform to a desired treatment regimen for the condition being treated. For example, blood pressure target thresholds could be set for each hour after symptom onset to achieve an ideal blood pressure profile over time.

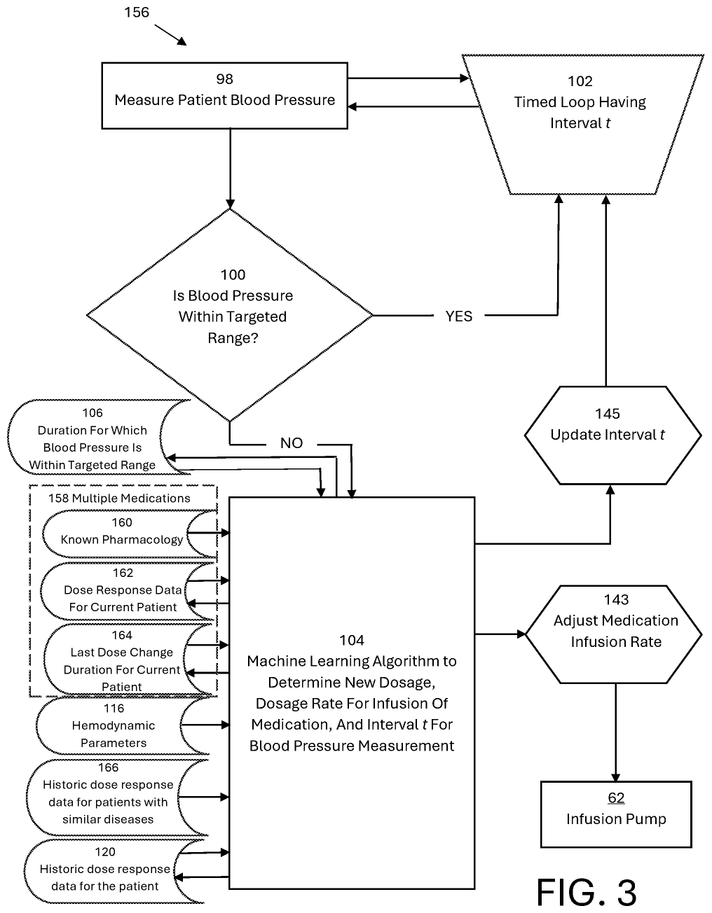

In FIG. 3, another embodiment of a process and algorithm for an artificial intelligence driven infusion pump system 156 is schematically depicted. This embodiment differs from that of FIG. 2 in that multiple medications are being dispensed. Such medications may be needed, for example, to target different aspects of the cardiovascular system or to avoid undesirable conditions that may result from the medications. Again, the first step is measurement of patient blood pressure 98, performed using blood pressure measuring device 32. The digitalized output of blood pressure measuring device 32 communicated to controller 23 may be internally validated to detect possible erroneous readings. Any suspected erroneous readings cause controller 23 to request a repeat measurement before proceeding.

Once an accurate blood pressure reading is achieved, controller 23 determines at step 100 whether the blood pressure reading is within a targeted range predetermined by the operator. For example, the operator could specify that systolic pressure must be between 100 and 140 mm/Hg. If yes, then blood pressure measuring device 32, which may operate on a timed loop 102 having variable time interval t, may be permitted to proceed with the next timed measurement of blood pressure at the set time interval t. Variable time interval t can be any suitable time period, but typically ranges from about 5 to 10 minutes.

Again, various data stores hosted in memory 26 can provide input to machine learning algorithm 104. Data store 106 contains the durations for which patient blood pressure has historically been within the operator specified targeted range, and is updated with each blood pressure measurement iteration. Data store 158 pertains to data regarding the multiple medications being dispensed, and generally includes known pharmacology 160 of the medications, historic dose response data 162 for each medication for the current patient, and last dose change duration 164 for the current patient. As depicted, dose response data 112 and last dose change duration 114 are updated with each blood pressure measurement iteration. As before, data store 116 can contain other hemodynamic parameters in addition to the primary hemodynamic parameters of blood pressure and heart rate such as SV, CO, and TPR. Data store 166 contains historic dose response data for all the medications being dispensed pertaining to patients with similar diseases, and may include a large database compiling such data. Data store 120 contains historic dose response data for the patient being treated, and can be updated with each blood pressure measurement iteration. It will be appreciated that, although not depicted in FIG. 2, measurements of intracranial pressure and/or heart rate may be supplied to controller 23 to enable machine learning algorithm 104 to take those parameters into account when determining an appropriate dosage recommendation. Data stores 106, 158, 160, 162, 164, 116, 166, and 120 are hosted in memory 26.

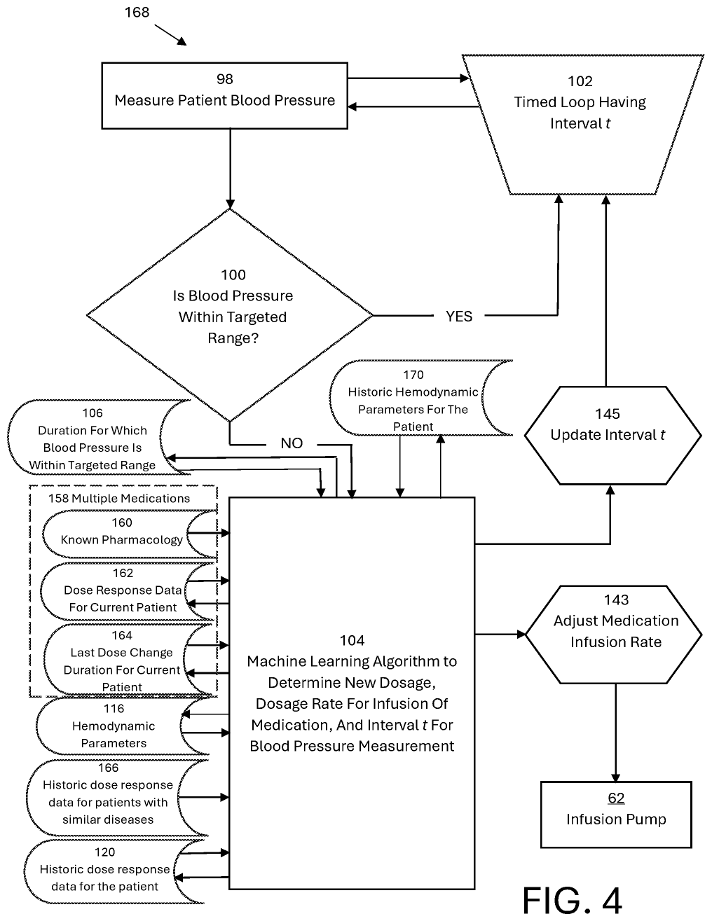

In FIG. 4, yet another embodiment of a process and algorithm for an artificial intelligence driven infusion pump system 168 is schematically depicted. This embodiment differs from that of FIGS. 2 and 3 in that hemodynamic parameters for the patient such as SV, CO, and TPR are stored over time and taken into account by machine learning algorithm 104. Again, the first step is measurement of patient blood pressure 98, performed using blood pressure measuring device 32. The digitalized output of blood pressure measuring device 32 communicated to controller 23 may be internally validated to detect possible erroneous readings. Any suspected erroneous readings cause controller 23 to request a repeat measurement before proceeding.

Once an accurate blood pressure reading is achieved, controller 23 determines at step 100 whether the blood pressure reading is within a targeted range predetermined by the operator. For example, the operator could specify that systolic pressure must be between 100 and 140 mm/Hg. If yes, then blood pressure measuring device 32, which may operate on a timed loop 102 having variable time interval t, may be permitted to proceed with the next timed measurement of blood pressure at the set time interval t. Variable time interval t can be any suitable time period, but typically ranges from about 5 to 10 minutes.

Again, various data stores hosted in memory 26 can provide input to machine learning algorithm 104. Data store 106 contains the durations for which patient blood pressure has historically been within the operator specified targeted range, and is updated with each blood pressure measurement iteration. Data store 158 pertains to data regarding the multiple medication being dispensed, and generally includes known pharmacology 160 of the medications, historic dose response data 162 for each medication for the current patient, and last dose change duration 164 for the current patient. As depicted, dose response data 112 and last dose change duration 114 are updated with each blood pressure measurement iteration. Data store 116 contains the primary hemodynamic parameters of blood pressure and heart rate, and data store 170 contains historic hemodynamic parameters SV, CO, and TPR. Data store 166 contains historic dose response data for all the medications being dispensed pertaining to patients with similar diseases, and may include a large database compiling such data. Data store 120 contains historic dose response data for the patient being treated, and can be updated with each blood pressure measurement iteration. Data stores 106, 158, 160, 162, 164, 116, 166, 168, and 120 are hosted in memory 26.

Hence, embodiments of the inventions described herein provide a compact system that can be arranged at the bedside of a patient and incorporate a dynamic and flexible artificial intelligence driven, machine learning algorithm for managing infusion of medications to manage patient blood pressure, particularly in challenging cases such as intracerebral hemorrhage. The algorithm can draw on large stores of data regarding the medications being dispensed, historic treatment of patients with the same or similar disease, and data pertaining to dose responsiveness of the current patient. This large mass of data can be efficiently analyzed by the algorithm to develop dosage recommendations, and dosage and blood pressure change data for consecutive dose adjustments can be archived, thereby combining automated blood pressure measurements with dose changes to develop new algorithms for dose recommendations. Internal validation compares actual blood pressure responses to dose adjustments with expected responses before altering the pre-existing algorithm to a new one. The system can enable input of multiple target blood pressures within a single cycle, corresponding to specific timeframes. Utilizing a time-based algorithm, it adjusts dosage to reach target blood pressures at different intervals post-disease onset, aiming to minimize mortality or disability based on trajectory analysis of blood pressure over 24 hours and previous patient studies.

Operators can accept recommended doses by selecting an icon on a touch screen interface, triggering feedback to the automated infusion pump for implementation. In some embodiments, the system receives blood pressure data, analyzes it to determine the recommended initiation or adjustment of intravenous medication dosage, and subsequently directs the automated infusion pump to administer the prescribed dose autonomously, without requiring input from medical professionals. The system may have inputs from multiple sensors to enable multiple infusion pumps to be controlled via multiple machine learning algorithms to enable simultaneous titration of multiple medications that target different aspects of cardiovascular systems.

The algorithm can include a PID approach. Such an algorithm operates on the principles of a PID control system, leveraging previous blood pressure values to dynamically adjust medication dosage and maintain blood pressure within a specified range. It incorporates patient-specific sensitivity to the administered drug, dynamically adjusting the dose step size as a proportional constant within the PID framework. By monitoring blood pressure over dynamic intervals and analyzing the patient's response to medication, the algorithm autonomously corrects deviations from the target range, optimizing medication dosage in real-time. This approach offers a sophisticated yet adaptable solution for hypertensive patient management, ensuring precise and personalized treatment while minimizing the risk of adverse effects.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

What is claimed is:

1. An automated system for managing blood pressure of a patient comprising:
a blood pressure measuring device;
a controller communicatively coupled with the blood pressure measuring device, the controller comprising:
a processor programmed with a machine learning algorithm, the algorithm adapted to receive a systolic blood pressure measurement from the blood pressure measuring device, perform a probabilistic determination to predict a blood pressure response to a particular dose of an intravenous medication and develop a dosage recommendation to achieve a predetermined target systolic blood pressure goal;
a memory adapted to store a plurality of data elements including data relating to the intravenous medication, data relating to the patient, data relating to a plurality of hemodynamic parameters, and data relating to other patients with a same or a similar condition; and
a user interface including a display; and
an infusion pump apparatus communicatively coupled with the controller and comprising an infusion pump, a medication bag fluidly coupled with the infusion pump, and an intravenous port fluidly coupled with the infusion pump;
wherein the machine learning algorithm uses at least one of the plurality of data elements to develop the dosage recommendation, the controller presents the dosage recommendation to an operator using the user interface, and the controller instructs the infusion pump to implement the dosage recommendation.

2. The system of claim 1, wherein the data relating to the medication includes at least one of known pharmacology of the medication, historic dose response data for the current patient, and last dose change duration for the current patient.

3. The system of claim 1, wherein the data relating to a plurality of hemodynamic parameters includes at least one of patient heart rate, patient stroke volume, patient cardiac output, and patient total peripheral resistance.

4. The system of claim 1, wherein the machine learning algorithm automatically alters itself based on patient blood pressure response to the medication after a predetermined number of sequential dose adjustments, and develops the dosage recommendation according to the altered algorithm.

5. The system of claim 1, wherein the system further comprises a second infusion pump apparatus communicatively coupled with the controller, the second infusion pump apparatus comprising a second infusion pump and a second medication bag fluidly coupled with the second infusion pump, the first infusion pump apparatus dispensing a first medication and the second infusion pump apparatus dispensing a second medication different from the first medication, the algorithm being further adapted to perform a probabilistic determination to predict a blood pressure response to a particular dose of each of the first and second medications and develop a dosage recommendation for each of the first and second medications to achieve the predetermined target systolic blood pressure goal.

6. The system of claim 1, further comprising a heart rate sensor communicatively coupled to the controller.

7. The system of claim 1, further comprising an intracranial pressure sensor communicatively coupled to the controller.

8. The system of claim 1, wherein the display of the user interface is touch sensitive and presents icons enabling the operator to accept, reject, or adjust the dosage recommendation.

9. An apparatus for determining an optimal intravenous medication dosage to manage blood pressure in a patient, the apparatus comprising:
a blood pressure measuring device;
a controller communicatively coupled with the blood pressure measuring device, the controller comprising:
a processor programmed with a machine learning algorithm, the algorithm adapted to receive a systolic blood pressure measurement from the blood pressure measuring device, perform a probabilistic determination to predict a blood pressure response to a particular dose of an intravenous medication and develop a dosage recommendation to achieve a predetermined target systolic blood pressure goal;
a memory adapted to store a plurality of data elements including data relating to the intravenous medication, data relating to the patient, data relating to a plurality of hemodynamic parameters, and data relating to other patients with a same or a similar condition; and a user interface including a display;
wherein the machine learning algorithm uses at least one of the plurality of data elements to develop the dosage recommendation, and the controller presents the dosage recommendation to an operator using the user interface.

10. The apparatus of claim 9, further comprising an infusion pump apparatus communicatively coupled with the controller and comprising an infusion pump, a medication bag fluidly coupled with the infusion pump, and an intravenous port fluidly coupled with the infusion pump, wherein the controller instructs the infusion pump to implement the dosage recommendation.

11. The apparatus of claim 10, wherein the data relating to the medication includes at least one of known pharmacology of the medication, historic dose response data for the current patient, and last dose change duration for the current patient.

12. The apparatus of claim 10, wherein the data relating to a plurality of hemodynamic parameters includes at least one of patient heart rate, patient stroke volume, patient cardiac output, and patient total peripheral resistance.

13. The apparatus of claim 10, wherein the machine learning algorithm automatically alters itself based on patient blood pressure response to the medication after a predetermined number of sequential dose adjustments, and develops the dosage recommendation according to the altered algorithm.

14. The apparatus of claim 10, wherein the apparatus further comprises a second infusion pump apparatus communicatively coupled with the controller, the second infusion pump apparatus comprising a second infusion pump and a second medication bag fluidly coupled with the second infusion pump, the first infusion pump apparatus dispensing a first medication and the second infusion pump apparatus dispensing a second medication different from the first medication, the algorithm being further adapted to perform a probabilistic determination to predict a blood pressure response to a particular dose of each of the first and second medications and develop a dosage recommendation for each of the first and second medications to achieve the predetermined target systolic blood pressure goal.

15. The apparatus of claim 10, further comprising a heart rate sensor communicatively coupled to the controller.

16. The apparatus of claim 10, further comprising an intracranial pressure sensor communicatively coupled to the controller.

17. The apparatus of claim 10, wherein the display of the user interface is touch sensitive and presents icons enabling the operator to accept, reject, or adjust the dosage recommendation.

18. A method of dispensing intravenous medication comprising:
measuring a patient blood pressure;
communicating the measured patient blood pressure to a controller having a processor programmed with a machine learning algorithm, the algorithm adapted to receive the measured patient blood pressure, perform a probabilistic determination to predict a blood pressure response to a particular dose of an intravenous medication and develop a dosage recommendation to achieve a predetermined target systolic blood pressure goal, the controller having a memory adapted to store a plurality of data elements including data relating to the intravenous medication, data relating to the patient, data relating to a plurality of hemodynamic parameters, and data relating to other patients with a same or a similar condition, wherein the machine learning algorithm uses at least one of the plurality of data elements to develop the dosage recommendation;
presenting the dosage recommendation to an operator through a user interface including a display; and
implementing the dosage recommendation with a first infusion pump apparatus communicatively coupled with the controller.

19. The method of claim 18, further comprising presenting the operator with controls enabling the operator to accept, reject, or alter the dosage recommendation.

20. The method of claim 18, further comprising providing a second infusion pump apparatus communicatively coupled with the controller, the first infusion pump apparatus dispensing a first medication and the second infusion pump apparatus dispensing a second medication different from the first medication, the algorithm being further adapted to perform a probabilistic determination to predict a blood pressure response to a particular dose of each of the first and second medications and develop a dosage recommendation for each of the first and second medications to achieve the predetermined target systolic blood pressure goal, and implementing each of the dosage recommendations for the first and second medications.
